(19) United States
(12) Patent Application Publication
(10) Pub. No.: US 2015/0289751 A1
(43) Pub. Date: Oct. 15, 2015

(54) Polymeric Articles Having a Nanoscopically and/or Microscopically Rough Surface, Methods of Use thereof, and Methods for Fabrication Thereof

(71) Applicant: UNIVERSITY OF UTAH RESEARCH FOUNDATION, Salt Lake City, UT (US)

(72) Inventors: Micah J. Frerck, Salt Lake City, UT (US); Alex J. Drew, Salt Lake City, UT (US); Branden D. Rosenhan, Salt Lake City, UT (US)

(73) Assignee: University of Utah Research Foundation, Salt Lake City, UT (US)

(21) Appl. No.: 14/439,858

(22) PCT Filed: Oct. 31, 2013
(86) PCT No.: PCT/US13/67751

§ 371 Date: Apr. 30, 2015

Related U.S. Application Data
(60) Provisional application No. 61/720,877, filed on Oct. 31, 2012.
(60) Provisional application No. 61/720,896, filed on Oct. 31, 2012.

(51) Int. Cl. A61B 1/00; B08B 17/06

(52) U.S. Cl. CPC A61B 1/00135; B08B 17/065; A61B 1/00096; A61B 1/0011

(57) ABSTRACT

Polymeric articles that include a nanoscopically and/or microscopically rough surface formed on at least a portion of the article. Methods of using and making such articles are also disclosed. In one embodiment, covers for use with a surgical viewing instrument that include the nanoscopically and/or microscopically rough surface are disclosed. The cover includes a cover configured for placement over at least a portion of a surgical viewing instrument, at least a portion of the cover being transparent to light, and a nanoscopically and/or microscopically rough surface formed on at least a portion of the cover. The nanoscopically and/or microscopically rough surface is configured to shed and/or incorporate into a thin, substantially uniform film such droplets, fogs, and debris for providing clearer and less obstructed vision at a surgical, diagnostic or procedure site.

### CROSS-REFERENCE TO RELATED APPLICATIONS

This application claims the benefit of and priority to U.S. Prov. Pat. App. Ser. No. 61/720,877 filed 31 Oct. 2012 and U.S. Prov. Pat. App. Ser. No. 61/720,896 filed 31 Oct. 2012, the entireties of which are incorporated herein by reference.

### BACKGROUND

There are many consumer and industrial products made from clear polymer materials where good visibility and non-obscure light transmission are highly desirable. The surfaces of such materials often come into contact with a variety of agents, including moisture, water, oils, aqueous salt solutions, acid or base solutions, and chemicals dissolved or suspended in aqueous compositions or other liquids. Likewise, a temperature differential between a polymeric surface and a warm, moist surface can lead to fogging. In addition, freezing liquids, such as water, can result in frozen deposits that are strongly adhered to the polymer surfaces. Alternatively, elevated temperatures can accelerate deleterious processes, such as corrosion or surface leaching.

Examples of transparent polymer surfaces that can be negatively impacted by the adhesion of moisture or other materials that can obscure vision and light transmission include ski goggles, swimming goggles, glasses, windows, vehicle windshields, motorcycle fairings, camera lenses, waterproof enclosures for cameras or other viewing equipment, endoscopes, smartphone surfaces, tablet computer surfaces, and the like. Even opaque polymer surfaces may be worth protecting from adhesion by moisture, water, oils, solutions, liquids or ice.

One example area where it is desirable to maintain a clear surface free of debris and fogging is through the viewing and/or illumination window of a surgical viewing instrument. For example, laparoscopic surgery, also called minimally invasive surgery (MIS), band-aid surgery, or keyhole surgery, is a modern surgical technique in which operations in the abdomen are performed through small incisions (usually 0.5-1.5 cm) as opposed to the larger incisions needed in laparotomy. There are a number of advantages to the patient with laparoscopic surgery versus an open procedure. These include reduced pain due to smaller incisions and hemorrhaging, and shorter recovery time. Laparoscopic surgery includes operations within the abdominal or pelvic cavities, whereas keyhole surgery performed on the thoracic or chest cavity is called thoracoscopic surgery. Laparoscopic and thoracoscopic surgery belong to the broader field of endoscopy.

Laparoscopy uses a thin, lighted tube put through a cut (incision) in the belly to look at the abdominal organs or the female pelvic organs. Laparoscopy is typically used to find problems such as cysts, adhesions, fibroids, and infection, but can also be used to remove and modify organs. Tissue samples can be taken for biopsy through the tube (laparoscope). Laparoscopic surgery makes use of images displayed on TV monitors to magnify the surgical elements.

FIG. 1 illustrates an exemplary laparoscopic procedure 100, which utilizes a laparoscope 102 held by one hand 104 of the surgeon and a surgical tool 106 held by the other hand 108 of the surgeon. During surgery within an insufflated body cavity 110 of a patient, light emitted from the distal end 112 of the laparoscope 102 illuminates the surgical site and permits the working end 114 of surgical tool 106 to perform a desired procedure on tissue 116.

There are two types of laparoscopes: (1) telescopic rod lens system, which is usually connected to a video camera (single chip or three chip) and (2) digital laparoscope where a charge-coupled device is placed at the distal end of the laparoscope, eliminating the rod lens system. Also attached is a fiber optic cable system connected to a cold light source (halogen or xenon) to illuminate the operative field. The abdomen is usually insufflated, or essentially blown up like a balloon, with carbon dioxide gas. This elevates the abdominal wall above the internal organs like a dome to create a working and viewing space.

During surgical operations, bodily fluids, blood, condensation, and the like can adhere to the observation window of the laparoscope so as to distort, obscure and degrade visibility of the surgical site. To maintain good visibility, the window typically requires frequent cleaning One common solution is to pull out the laparoscope and wipe it with a cleaning cloth. Another is to wipe it on an internal organ such as the liver. Yet another involves keeping the laparoscope warm to prevent condensation, such using a scope heater prior to insertion into the body cavity. Some laparoscopes are equipped with cleaning mechanisms, such as a wiping system described in U.S. Pat. No. 7,959,561, a trocar or hub that cleans the laparoscope each time it is pulled out and reinserted through the trocar or hub, or air flow mechanism that prevents condensation by blowing air against the tip.

FIG. 2 illustrates the wiping system of U.S. Pat. No. 7,959,561, which includes a rigid endoscope 2, a washing sheath 3, and a wiper sheath 4. A portion of endoscope 2 is inserted through a sheath insert section of washing sheath 3, which is in turn inserted through a wiper insert section of wiper sheath 4. A cleaning liquid and water is fed through the washing sheath, which cooperates with the wiper sheath to clean an observation window of the endoscope 2 during use.

While physically cleaning the laparoscope is effective to ensure a clean and clear observation window, it requires the surgeon to remove the laparoscope from the surgical site and/or requires two hands. In either case, the surgery is interrupted, lengthening the time of the procedure. Self-cleaning mechanisms, though convenient and essentially automatic, are less effective in ensuring a clean and clear observation window (e.g., because they typically only prevent fogging or remove condensation but do not effectively remove blood and tissue debris).

### SUMMARY

Disclosed herein are polymeric articles that include a nanoscopically and/or microscopically rough surface formed on at least a portion of the article. Methods of using and making such articles are also disclosed. In one embodiment, covers for use with a surgical viewing instrument that include the nanoscopically and/or microscopically rough surface are disclosed. A variety of surgical viewing instruments such as, but not limited to, laparoscope, endoscopes, capsule endoscopes, pill cameras, and surgical microscopes are routinely used for visualizing and/or illuminating a medical procedure site. However, during surgical operations, bodily fluids, blood, condensation, and the like can adhere to the observation window of the surgical viewing instrument(s) and distort, obscure and degrade visibility of the surgical site. For example, the window can fog up due to the temperature differential between the surgical viewing instrument and the warm, humid environment in and around the body. In addition, smoke, blood, and tissue debris from ablation, cutting or cautery can adhere to the window. Spraying and smudging by blood and tissue debris can also obscure vision. The nanoscopically and/or microscopically rough surface is configured to shed and/or incorporate into a thin, substantially uniform film such droplets, fogs, and debris for providing clearer and less obstructed vision at a surgical, diagnostic or procedure site.

In an embodiment, a cover for use with a surgical viewing instrument for providing clearer and less obstructed vision at a surgical, diagnostic or procedure site is disclosed. The cover includes a cover configured for placement over at least a portion of a surgical viewing instrument, at least a portion of the cover being transparent to light, and a nanoscopically and/or microscopically rough surface formed on at least a portion of the cover that provides a clearer and less obstructed view of a surgical, diagnostic or procedure site through the surgical viewing instrument. Also disclosed are methods of making and using such covers.

According to one embodiment, the cover includes an elongate tubular member that at least partially encloses a surgical viewing instrument (e.g., a laparoscope) during use. The nanoscopically and/or microscopically rough surface may be positioned on the distal tip and/or sidewall of the elongate tubular member. The elongate tubular member may further include a rigid hub attached to a proximal end of the elongate tubular member to provide added strength and gripping ability.

Depending on surface chemistry, the nanoscopically and/or microscopically rough surface can be at least one of a highly hydrophobic, highly oleophobic, or highly hydrophilic surface. According to one embodiment, the nanoscopically and/or microscopically rough surface comprises a highly hydrophobic composition that repels water and other hydrophilic substances. The highly hydrophobic composition may comprise nanoparticles held to the cover by one or more types of silanes and preferably cross-linked to increase strength and prevent hydrolysis during use. The highly hydrophobic composition may further comprise a hydrophobic surface modifying agent to maximize hydrophobicity.

The highly hydrophobic composition advantageously repels water as a result of surface tension and preferential self-adhesion of water molecules to themselves rather than the highly hydrophobic composition. The highly hydrophobic composition can be formulated so as to cause water or protein-based droplets to have a high surface angle relative to the polymer surface of the cover (e.g., at least about 135°, 140° or 150°). The highly hydrophobic composition is formulated so as to cause water or protein-based droplets to have a low shedding angle or hysteresis angle relative to the sheath (e.g., less than about 30°, 15° or 10°). The highly hydrophobic composition can be formulated so that the cover does not decrease light transmittance through the cover by more than about 20%.

According to another embodiment, the nanoscopically and/or microscopically rough surface comprises a highly oleophobic composition that repels oils and other non-polar substances. The highly oleophobic composition may comprise nanoparticles held to the cover by one or more types of silanes and preferably cross-linked to increase strength and prevent hydrolysis during use. The highly hydrophobic composition may further comprise an oleophobic surface modifying agent (e.g., a fluorinated compound) to maximize oleophobicity.

According to yet another embodiment, the nanoscopically and/or microscopically rough surface comprises a highly hydrophillic composition that may readily absorb water and water-based liquids and thereby form a substantially uniform aqueous layer on the cover. Such a substantially uniform aqueous layer may, for example, prevent fogging. Such a highly hydrophilic composition may also be highly oleophobic. The highly hydrophilic composition may comprise nanoparticles held to the cover by one or more types of silanes and preferably cross-linked to increase strength and prevent hydrolysis during use. The highly hydrophilic composition may further comprise a hydrophilic surface modifying agent (e.g., a polyethylene glycol compound) to maximize hydrophilicity.

An exemplary a method of performing a laparoscopic procedure comprises: (1) positioning a nanoscopically and/or microscopically rough surface on at least a viewing and illumination portion of a surgical viewing instrument; (2) positioning the laparoscope at a surgical site; and (3) utilizing the laparoscope to illuminate and view the surgical site, (4) the nanoscopically and/or microscopically rough surface reducing or preventing adhesion of substances that obstruct vision at the surgical site. According to one embodiment, positioning the nanoscopically and/or microscopically rough surface comprises placing a sheath (e.g., an elongate tubular member) carrying the nanoscopically and/or microscopically rough surface over at least a portion of the laparoscope. According to another embodiment, positioning the nanoscopically and/or microscopically rough surface comprises placing a transparent film (e.g., an adhesive strip) carrying the nanoscopically and/or microscopically rough surface over at least a portion of the laparoscope.

An exemplary method for manufacturing a cover for use with a surgical viewing instrument for providing clearer and less obstructed vision at a surgical site comprises: (1) providing a polymeric member configured for placement over at least a portion of a surgical viewing instrument, at least a portion of the cover being transparent to light; and (2) forming a nanoscopically and/or microscopically rough surface on at least a portion of the polymeric member, wherein the nanoscopically and/or microscopically rough surface reduces or prevents adhesion of substances that obstruct vision at a surgical site.

According to one embodiment, forming the nanoscopically and/or microscopically rough surface on the polymeric member comprises: (1) reacting an organic binder with functional groups on the polymer surface to bond organic binder molecules to the polymer surface; (2) reacting nanoparticles with the organic binder molecules; (3) reacting a cross-linking agent with the nanoparticles to form cross-linked nanoparticles; and (4) applying a surface modifying agent to the cross-linked nanoparticles. The polymer surface and intermediate modified polymer surfaces can be activated using plasma or chemical activation.

According to another embodiment, forming the nanoscopically and/or microscopically rough surface on the polymeric member comprises: (1) forming a nanoscopically and/or microscopically roughened surface on at least one surface of a mold configured for molding a polymeric member; and (2) molding the polymeric member in the mold, wherein, in the molding, the nanoscopically and/or microscopically rough surface is imprinted on the polymeric member.

In addition to the foregoing described covers for surgical viewing instruments, the nanoscopically and/or microscopically rough surface may be formed on a number of articles including, but not limited to, ski goggles, swimming goggles, glasses, windows, vehicle windshields, motorcycle fairings, camera lenses, waterproof enclosures for cameras or other viewing equipment, smartphone surfaces, tablet computer surfaces, and the like. The nanoscopically and/or microscopically rough surfaces described herein can protect a surface from moisture, water, oils, solutions, liquids, ice or other materials.

These and other benefits, advantages and features of the present invention will become more fully apparent from the following description and appended claims, or may be learned by the practice of the invention as set forth hereinafter.

### BRIEF DESCRIPTION OF THE DRAWINGS

In order that the manner in which the above recited and other benefits, advantages and features of the invention are obtained, a more particular description of the invention briefly described above will be rendered by reference to specific embodiments thereof which are illustrated in the appended drawings. The following drawings depict only typical embodiments of the invention and are not therefore to be considered limiting of its scope:

FIG. 1 illustrates an exemplary laparoscopic procedure at a surgical site;

FIG. 2 illustrates an exemplary endoscope wiping system for physically cleaning an endoscope during an endoscopic procedure;

FIG. 3 is flow chart illustrating an exemplary method of applying a highly hydrophobic coating to a polymer surface of a laparoscopic cover;

FIGS. 4A-4E is a diagram illustrating an exemplary reaction sequence for forming a superhydrophobic coating on a surface;

FIGS. 5A-5C illustrate an exemplary embodiment of an endoscope cover for use with an endoscope in an endoscopic procedure;

FIGS. 6A-6B schematically illustrate water droplets on a surface and having different contact angles;

FIG. 7 schematically illustrates the hysteresis angle of a droplet of water on a surface; and

FIGS. 8A and 8B illustrate exemplary coating materials with superhydrophobic and superhydrophilic regions.

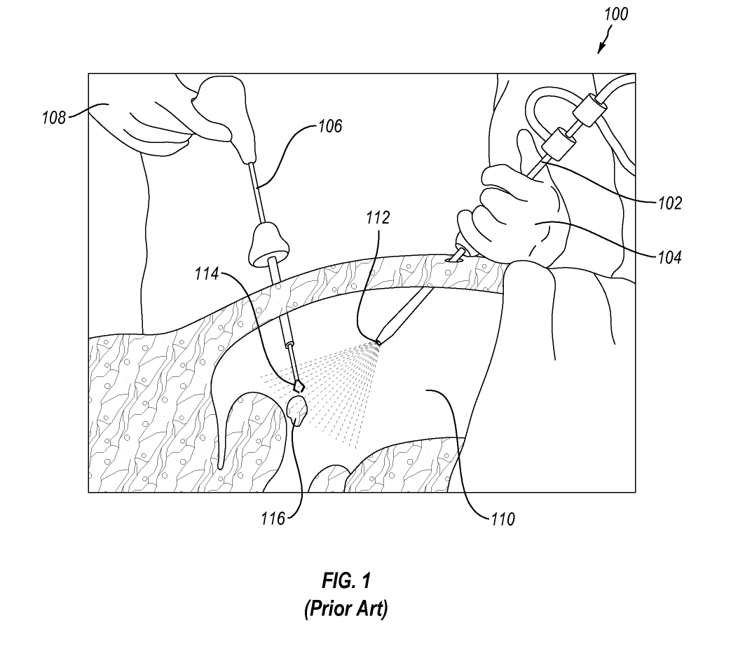

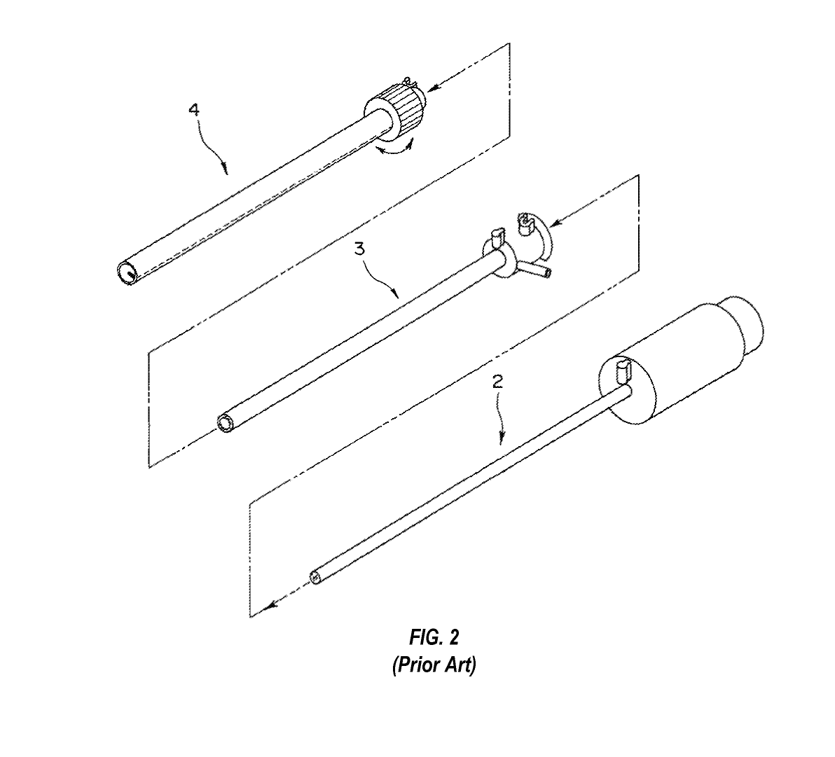

### DETAILED DESCRIPTION

Disclosed herein are polymeric articles that include a nanoscopically and/or microscopically rough surface formed on at least a portion of the article. Methods of using and making such articles are also disclosed. In one embodiment, covers for use with a surgical viewing instrument that include the nanoscopically and/or microscopically rough surface are disclosed. A variety of surgical viewing instruments such as, but not limited to, laparoscope, endoscopes, capsule endoscopes, pill cameras, and surgical microscopes are routinely used for visualizing and/or illuminating a medical procedure site. However, during surgical operations, bodily fluids, blood, condensation, and the like can adhere to the observation window of the surgical viewing instrument(s) and distort, obscure and degrade visibility of the surgical site. For example, the window can fog up due to the temperature differential between the surgical viewing instrument and the warm, humid environment in and around the body. In addition, smoke, blood, and tissue debris from ablation, cutting or cautery can adhere to the window. Spraying and smudging by blood and tissue debris can also obscure vision. The nanoscopically and/or microscopically rough surface is configured to shed and/or incorporate into a thin, substantially uniform film such droplets, fogs, and debris for providing clearer and less obstructed vision at a surgical, diagnostic or procedure site.

As used herein, the term “surgical viewing instrument” shall include all instruments that are used for viewing and/or illuminating a surgical procedure site. Typical surgical viewing instruments include, but are not limited to, laparoscope, endoscopes, capsule endoscopes, pill cameras, and surgical microscopes. In some embodiments, laproscopes and endoscopes are referred to specifically. As used herein, the terms “laparoscope” and “endoscope” shall have their ordinary meaning In addition, the term “endoscope” shall broadly mean any “boroscope” unless otherwise limited. Examples of endoscopes include, but are not limited to, flexible and rigid arthroscopes, bronchoscopes, colonoscopes, cystoscopes, enteroscopes, esophagogastroduodenoscopes, hysteroscopes, laparoscopes, laryngoscopes, mediastinoscopes, sigmoidoscopes, or thoracoscopes. The covers described herein may be adapted for fit essentially any laproscope. For example, some laproscopes use a lens that is angled (e.g., 30°, 45°, etc.) relative to the elongate stem of the laproscope. Likewise, some laproscopes use a lens that is articulated and variably anglable relative to the elongate stem of the laproscope. The cover described herein may be adapted to such devices. For the sake of brevity, the term “laparoscope” is often used; however, the disclosure will apply to endoscopes and boroscopes generally unless otherwise limited.

As used herein, the term “nanoscopically and/or microscopically” refers to the fact that the surface(s) described herein may be nanoscopically or microscopically rough (i.e., the surface may include either nanoscopic or microscopic surface features) or nanoscopically and microscopically (i.e., the surface may include both nanoscopic and microscopic surface features).

The surgical viewing instrument covers described herein are especially useful in maintaining clearer and less obstructed vision in combination with a telescopic rod lens system comprised of a camera and light source. The covers have anti-adhesion properties in order to prevent or reduce adhesion of bodily fluids, blood, condensation, smoke, tissue debris and the like and/or prevent fogging and smudging. Maintaining a clearer and less obstructed vision reduces or eliminates the need to physically clean the instrument during a procedure, which can reduce the time and effort required to complete the procedure.

According to one embodiment, the inventive covers include a sheath configured for placement over at least a portion of a surgical viewing instrument and a nanoscopically and/or microscopically rough surface formed on or applied to at least a portion of the sheath. At least a portion of the sheath is advantageously transparent to light so as to act as a window through which light can pass. The nanoscopically and/or microscopically rough surface (e.g., a highly hydrophobic coating as described more fully herein) reduces or prevents adhesion of substances that obstruct vision at a surgical site, such as bodily fluids, blood, condensation, smoke, tissue debris and the like and/or prevents fogging and smudging.

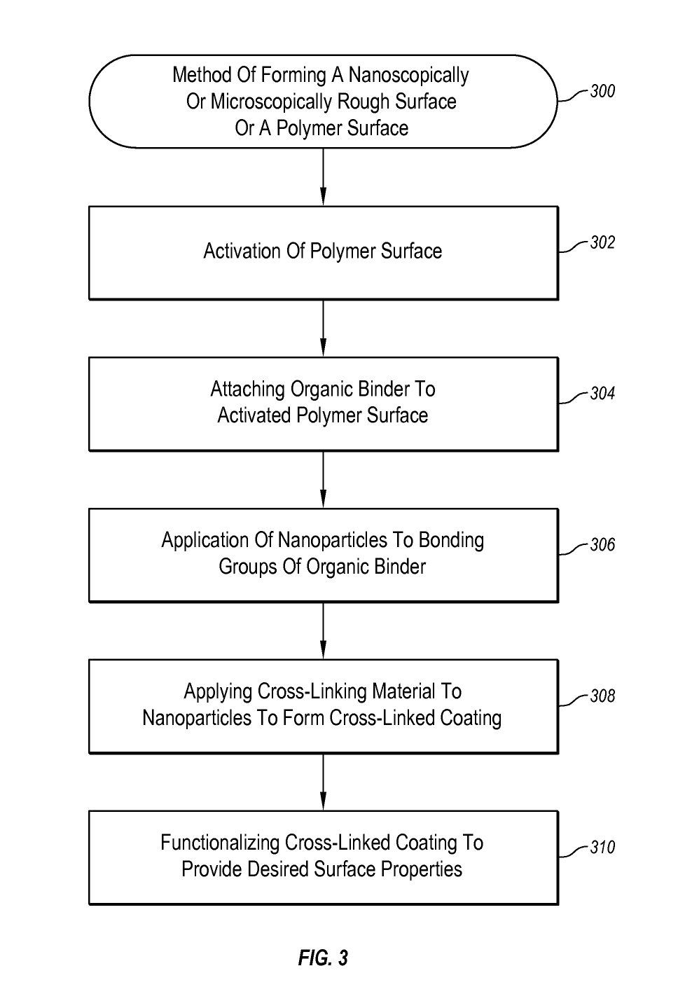

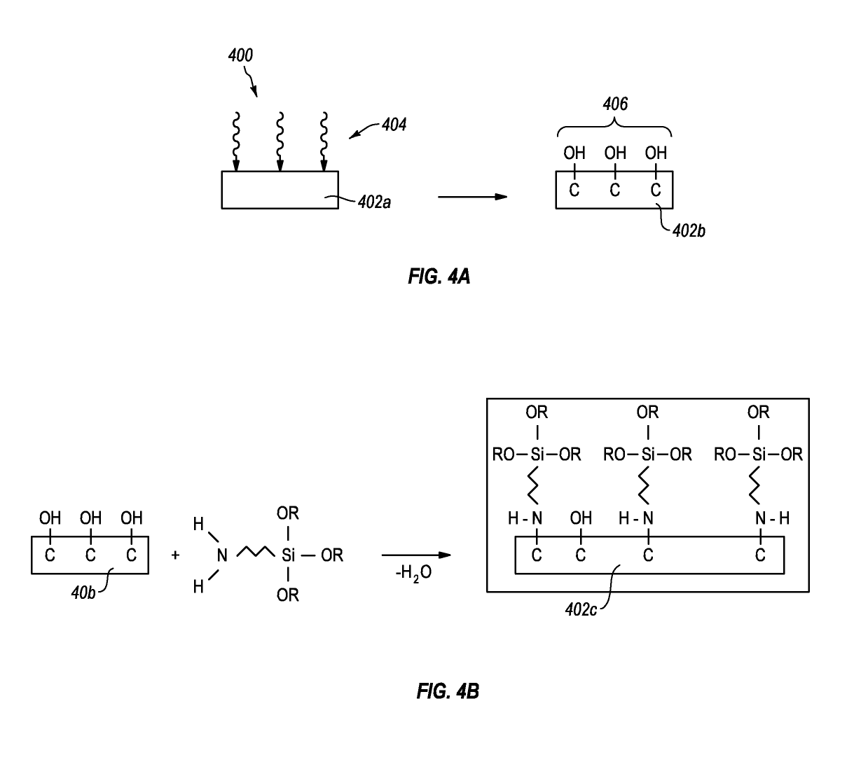

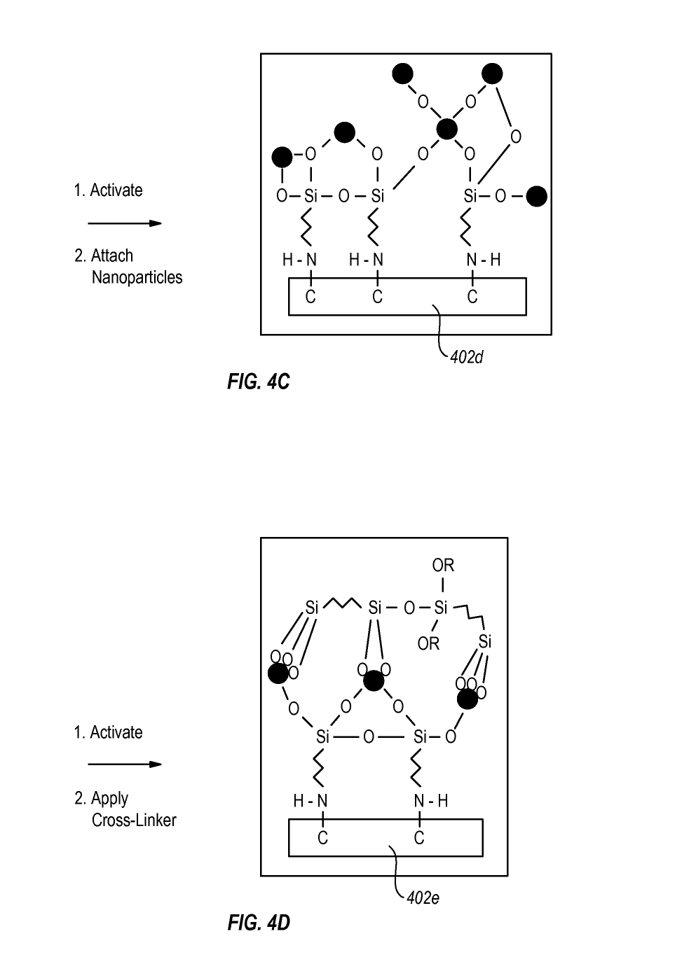

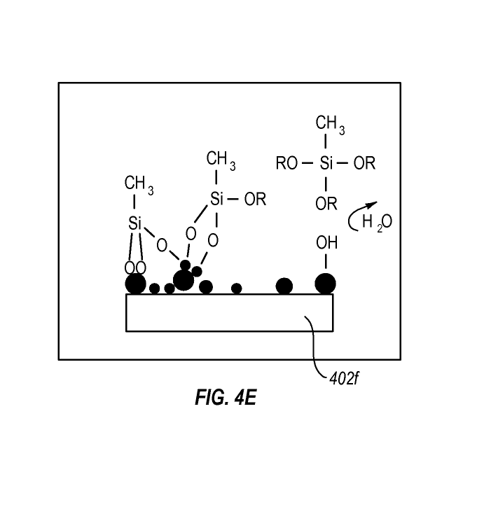

FIG. 3 is a flow chart that illustrates an exemplary method 300 for forming a nanoscopically and/or microscopically rough surface on a polymer surface. In one example, the polymer surface that includes the nanoscopically and/or microscopically rough surface may include at least a portion of a laparoscope cover. FIGS. 4A-4E provide additional details of an exemplary method for forming the nanoscopically and/or microscopically rough surface on a polymer surface.

In a first step 302, the polymer surface is activated to create or expose functional groups with which binder molecules can react. Activation can be achieved using any polymer surface activation method known in the art. One example of polymer surface activation is plasma (or corona) activation using radiant energy. Another example of polymer surface activation is chemical activation, such as by using a solvent, acid or base.

In subsequent step 304, an organic binder is attached to the activated polymer surface. This may be performed by chemically reacting organic binder molecules with the functional groups on the polymer surface. The organic binder molecules advantageously include reactive groups that are able to react with functional groups on the polymer surface, such as by a condensation or substitution reaction. According to one embodiment, and by way of example only, the functional groups on the polymer surface may include hydroxyl groups and the reactive groups of the organic binder molecules are amine groups capable of displacing the hydroxyl groups from the polymer surface to form an amine bond with carbon atoms on the polymer surface. If water is formed as a byproduct, the intermediate product may be dried prior to performing the next step.

In subsequent step 306, nanoparticles and/or microparticles are applied to and reacted with bonding groups of the organic binder molecules. The nanoparticles and/or microparticles provide nanoscale roughness to the nanoscopically and/or microscopically rough surface to, for example, increase the bond angle of water to the coating surface beyond whatever bond angle can be achieved by the hydrophobicity of a highly hydrophobic coating by itself. According to one embodiment, the organic binder molecules (e.g., amino silane molecules) are quick activated (e.g., heated) using plasma activation. In the case of fumed silica or titanium dioxide particles, such particles can be sonicated in ethanol and pH stabilized (e.g., using an acid, such as acetic acid) to deagglomerate the nanoparticles to provide a colloidal mixture prior to application to the organic binder treated polymer surface. If water is formed as a byproduct, the intermediate product may be dried prior to performing the next step.

In subsequent step 308, a cross-linking material is applied to the nanoparticles to increase strength and prevent hydrolysis of the nanoparticles from the binding agent during use. The nanoparticles can be quick activated using plasma activation. According to one embodiment, steps 306 and 308 may be combined in a single reaction vessel although two-step application and cross-linking of the nanoparticles is currently preferred.

In subsequent step 310, the cross-linked nanoparticle surface of the coating may be quick activated using plasma activation and then functionalized using one or more desired functionalizing agents. According to one embodiment, the functionalizing agent may be a hydrophobic material, such as a fluoroalkyl or silane material, which yields a highly hydrophobic coating. Adding a hydrophobic functionalizing agent serves to maximize hydrophobicity of the highly hydrophobic coating. According to another embodiment, if oleophobicity is desired, at least a portion of the cross-linked nanoparticle surface may be treated with an oleophobic functionalizing agent, such as a fluoroalkyl or another highly polar functionalizing agent. Some oleophobic materials (e.g., fluoroalkyls) may be both hydrophobic and oleophobic. According to another embodiment, a portion of the cross-linked nanoparticle surface may be treated with a hydrophilic functionalizing agent, such as polyethylene glycol (PEG) and the like. Providing hydrophobic, oleophobic, and/or hydrophilic agents yields nanoscopically and/or microscopically rough surface having desired properties for preventing adhesion of vision obscuring substances to the cover.

FIGS. 4A-4E show a reaction sequence that more particularly illustrates examples of materials and reactions used to form a nanoscopically and/or microscopically rough surface on a polymer surface, such as a polymer surface of a cover for a surgical viewing instrument. FIG. 4A shows a process 400 in which an initial polymer surface 402a is activated using plasma activation 404 to yield a functionalized polymer surface 402b having functional groups 406 (illustrated as being hydroxyl groups, although it is possible to form other functional groups, such as carboxyl groups, amino groups, halide groups, sulfonyl groups, and the like). The polymer surface 402a can alternatively be activated chemically, such as by using a solvent, acid or base. The polymer substrate can be any polymer capable of forming functional groups through surface activation. Examples of suitable polymers include polycarbonates, polyethylene terephthalate glycol modified (PETG), polystyrene, and the like.

FIG. 4B shows a subsequent step of process 400 in which a functionalized polymer surface 402b having functional groups is reacted with an organic binder to yield a modified polymer surface 402c having organic binder molecules bonded thereto. An example of an organic binder is 3-(aminopropyl) triethoxy silane (APTES) as illustrated in FIG. 4B when R=ethyl. According to other embodiments, R can be propyl, butyl or larger). When R is ethyl or larger steric hindrance prevents formation of Si—O—C bonds with the polymer surface and favors formation of amine bonds. According to one embodiment, the reaction shown in FIG. 4B can be carried out by reacting the functionalized polymer surface 402b with a reaction mixture that includes APTES, water and an acid catalyst (e.g., 5% acetic acid). Because water is formed as a byproduct, this reaction may be considered to be a condensation reaction when the hydroxyl functional groups are displaced and combine with hydrogen atoms displaced from the amino reactive group of the APTES molecules. The modified polymer surface 402c having organic binder molecules bonded thereto is advantageously dried to remove water prior to the next step. While FIG. 4B illustrates amine bonds formed by displacing the hydroxyl groups, amide bonds can be formed where the polymer surface includes carboxyl group functionality.

FIG. 4C shows a subsequent step of process 400 in which a modified polymer surface 402c having organic binder molecules bonded thereto is quick activated (e.g., using plasma activation) and then coated with nanoparticles to form nanoparticle treated polymer surface 402d. In this embodiment, the nanoparticles can react with the organic binder molecules by displacing one or more leaving groups (e.g., the R groups from siloxane molecules, which form alcohol molecules as biproduct) to from bonds with the organic binder molecules. In this example, metal oxide nanoparticles can form Me-O—Si bonds with the siloxane molecules. In the case of fumed silica, Si—O—Si bonds are formed. Examples of other nanoparticle materials include titanium dioxide, alumina, and nanoclays. The intermediate nanoparticle treated polymer surface 402d is advantageously dried prior to the next step.

FIG. 4D shows a subsequent step of process 400 in which a nanoparticle treated polymer surface 402d is quick activated (e.g., using plasma activation) and then reacted with a cross-linking agent to form cross-linked nanoparticle treated polymer surface 402e. In this embodiment, the cross-linking agent may comprise a dipodal silane, such as bis-triethoxy-silyl ethane (BTESE). The ethoxy groups act as leaving groups when BTESE reacts with surface hydroxyl groups of the nanoparticles, yielding ethanol as byproduct. The cross-linked nanoparticle treated polymer surface 402e is advantageously dried prior to the next step.

FIG. 4E shows a subsequent step of process 400 in which a cross-linked nanoparticle treated polymer surface 402e is quick activated (e.g., using plasma activation) and then reacted with a functionalizing agent to yield a non-stick polymer surface 402f having desired properties. According to the illustrated embodiment, the functionalizing agent comprises methyltriethoxysilane (MTES). The ethoxy groups act as leaving groups when MTES reacts with surface hydroxyl groups of the nanoparticles, yielding ethanol as byproduct. Additional examples of hydrophobic functionalizing agents include, but are not limited to, methyltriethoxysilane, dimethdiethoxysilane, octyltriethoxysilane, hexadecyltriethoxysilane, octadecyltriethoxysilane, isobutyltriethoxysilane, nonafluorohexyltriethoxysilane, tridecafluoro-1,1,2,2-terahydoocyl)triethoxysilane, heptadecafluoro-1,1,2,2-terahydoocyl)triethoxysilane, and diphenyldiethoxysilane. Alternatives include trimethoxy, and trichloro versions of the above mentioned compounds. The functionalizing agent may alternatively include fluoroalkyl groups to provide a nanoscopically and/or microscopically rough surface that is both hydrophobic and oleophobic. The methyl group of MTES may be replaced with a longer alkyl group to provide desired surface properties. According to one embodiment, a portion of the cross-linked nanoparticle treated polymer surface 402e may be treated with a hydrophilic functionalizing agent (e.g., PEG) in order to provide hydrophilic properties in specified regions when desired. Additional examples of hydrophilic functionalizing agents include, but are not limited to, N-(3-triethoxysilylpropyl)-O-polyethylene-oxide urethane, Bis(3-methyldimethoxysilyl)propyl)-propylene oxide, and N-N-BIS-[(3-triethoxysilylpropyl)amino-carbonyl]polyethylene oxide (ureasil).

In general, a method for forming a nanoscopically and/or microscopically rough surface with superhydrophobic, superoleophobic, or superhydrophilic surface or combination therein involves activating a substrate to allow for the chemical bonding of an adhesion intermediate, whereby a deposition of nanoparticles and/or microparticles can be utilized to construct a roughened surface. The roughened surface can be cross-linked to ensure greater mechanical durability and may subsequently be coated with a secondary material to improve hydrophobicity, oleophobicity, and/or hydrophilicity or a combination therein.

An exemplary method for forming a superhydrophobic, superoleophobic, and/or superhydrophilic surface or combination therein comprises the steps of: (1) activating a substrate to improve chemical bonding; (2) depositing an adhesion promoter; (3) depositing nanoparticles to said surface to create a nanoscopically and/or microscopically rough surface; and (4) depositing a hydrophobic, oleophobic, and/or hydrophilic material to improve hydrophilicity, oleophobicity, or hydrophobicity or combination thereof.

According to one embodiment, a superhydrophobic surface is provided wherein the water contact angle is greater than or equal to 150 degrees and/or wherein the sliding contact angle is less than or equal to 10 degrees. In another embodiment, a superhydrophilic surface is provided wherein the water contact angle is less than or equal to 10 degrees.

According to another embodiment, a superoleophobic surface is provided. According to one embodiment, a superoleophobic surface can be defined as follows: (1) A surface with nanoscopic and or microscopic roughness; (2) a surface with a static nonpolar solution contact angle greater than or equal to 150°; and (3) surface with a hysteresis angle/sliding angle less than or equal to 10°.

According to another embodiment, a superhydrophillic surface is provided. A superhydrophillic surface can be defined as follows: (1) A surface with nanoscopic and or microscopic roughness with a static water or polar solution contact angle less than or equal to 0°.

With regard to activation of a substrate, the activation process can be performed using an oxygen plasma, a corona discharge, or using chemical means, such as a solvent, oxidizer, acid, or base.

According to one embodiment, the substrate may be comprised of an organic material. Examples include thermoset polymers, thermoplastic polymers, epoxies, furans, polyimides, melamines, polyesters, and urethanes. Examples of thermoplastic polymers include, but are not limited to, acrylonitrile butadiene styrene (ABS), celluloid, cellulose acetate, ethylene-vinyl acetate (EVA), poly(methyl methacrylate) (PMMA), polyacrylonitrile (PAN), polyamide (Nylon), polycarbonate (PC), polyether ether ketone (PEEK), polyethylene (PE), poly(ethylene terephthalate) (PET), polypropylene (PP), polystyrene (PS), polysulfone, polyvinyl chloride (PVC), styrene-acrylonitrile, polydimethylsiloxane, and combinations thereof.

According to another embodiment, the substrate may be comprised of an inorganic material. Examples include, but are not limited to, glass, silica, quartz, talc, mica, clay, aluminum, iron, steel, and combinations thereof.

According to one embodiment, the adhesion promoter can be a silane comprised of an inorganic and organic functionality with one (R₃—Si—OR), two (R₂—Si—(OR)₂), or three (R₂—Si—(OR)₃) alkoxy groups. Examples of functional groups of the adhesion promoter groups include ethyl, propyl, or butyl alkoxy group. A larger end group decreases the rate of hydrolysis and formation of silanols and moreover sterically hinders alcoholysis with substrate hydroxyl groups. Advantageously, the organic functional group of the adhesion promoter is tailored to react with the specified substrate. According to one embodiment, the adhesion promoter forms a 0.1-5% solution in water adjusted to 4.5-5.5 pH with acetic acid. In another embodiment, the adhesion promoter forms a 0.1-5% solution in an aqueous mixture of 95% methanol, ethanol, or isopropanol with 5% water adjusted to 4.5-5.5 pH with acetic acid. In yet another embodiment, the adhesion promoter can be amino functionalized and form a 0.1-5% solution in water or a 0.1-5% solution in an aqueous mixture of 95% methanol, ethanol, or isopropanol with 5% water. According to another embodiment, the adhesion promoter can be deposited using a dilute solvent such as, but not limited to, methyl ethyl ketone (MEK), acetone, and/or 2-butanol, in order to facilitate in the development of an interpenetrating network between the substrate and the adhesion promoter. According to one embodiment, the adhesion promoter can be deposited under 10-30 PSI of pressure to facilitate in the development of an interpenetrating network between the substrate and the adhesion promoter. Moreover, the adhesion promoter can be deposited under an elevated temperature of 50-80° C. to facilitate in the development of an interpenetrating network between the substrate and the adhesion promoter. The adhesion promoter can be cured for 20-60 minutes at 50-110° C. or 24 hours at room temperature.

According to one embodiment, the nanoparticles can have a diameter that ranges from 10 nm and 1 micron, preferably from 10 nm to 200 nm to achieve a transparent coating. Examples of nanoparticle materials include, but are not limited to, clays, talc, mica, silica, alumina, wollastonite, titanium dioxide, and combinations thereof.

According to one embodiment, the cross linker can be classified as a dipodal silane. In one embodiment, the cross-linker can be added to the substrate following deposition of nanoparticles. In an alternative embodiment, the cross linker can be combined at a 1:5 to 1:10 ratio with the nanoparticle deposition. Examples of cross linkers include, but are not limited to, bis(triethoxysilyl)ethane, bis(triethoxysilyl)octane, bis(trimethoxysilylethyl)benzene, bis[(3-methyldimethoxysilyl)propyl]-polypropylene oxide.

In an alternative method, the nanoscopically and/or microscopically rough surface may be formed on a polymeric surface using a deformation process. For example, a thermoplastic substrate is heated and strained but not beyond yield. The plastic is dipped in an appropriate adhesion promoter with an organofunctional group and three alkoxy groups. Tetraethylorthosilicate may be included to produce a sizable glass layer. When allowed to relax the glass layer should induce strain wrinkling and nanoscopic wrinkles

In another embodiment, the nanoscopically and/or microscopically rough surface may be formed on a polymeric surface using a molding process. For example, the aforementioned nanoscopically and/or microscopically rough surfaces may be formed on one or more surfaces of a mold, which can be subsequently be used to mold a plastic (e.g., by injection molding or thermosetting) to form a nanoscopically and/or microscopically rough surface on the polymer surface. Such a surface would not necessarily include nanoparticles and the like, but would instead include the impression of the nanoparticles. In another embodiment, a nanopore aluminum oxide template is produced and used to hot emboss a polymer substrate to achieve a nanoscopically roughened surface. Such surfaces may be highly hydrophobic, highly oleophobic, and/or highly hydrophilic depending in the type of polymer chose to form the polymer surface.

A nano/microscopically rough surface created by the electrospinning of polymeric fibers or droplets to form a surface with nano/microsized features.

Alternatively, the nanoscopically and/or microscopically rough surface may be formed on a polymeric surface by one or more of wet etching of the substrate with appropriate chemicals to form a surface with nano/microsized features, dry etching the substrate with plasmas, reactive ions, or corona discharge to form a surface with nano/microsized features, by a layer-by-layer deposition of varying polyanions and polycations to form a surface with nano/microsized features, by chemical vapor deposition of reactants to form a surface with nano/microsized features, by a sol-gel deposition of a polycondensed network to form a surface with nano/microsized features, or by phase separation of a polymer to form a surface with nano/microsized features.

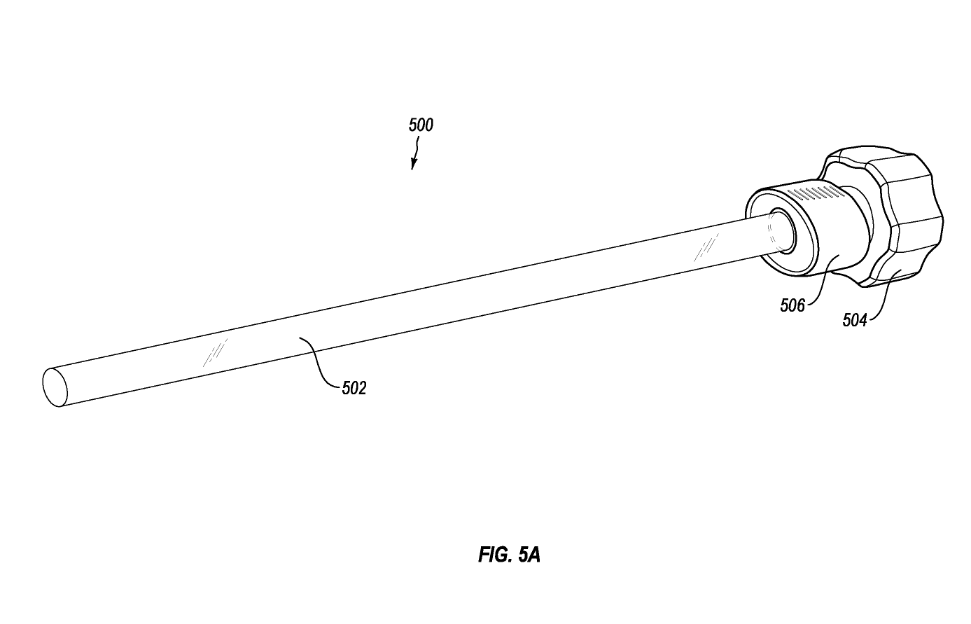

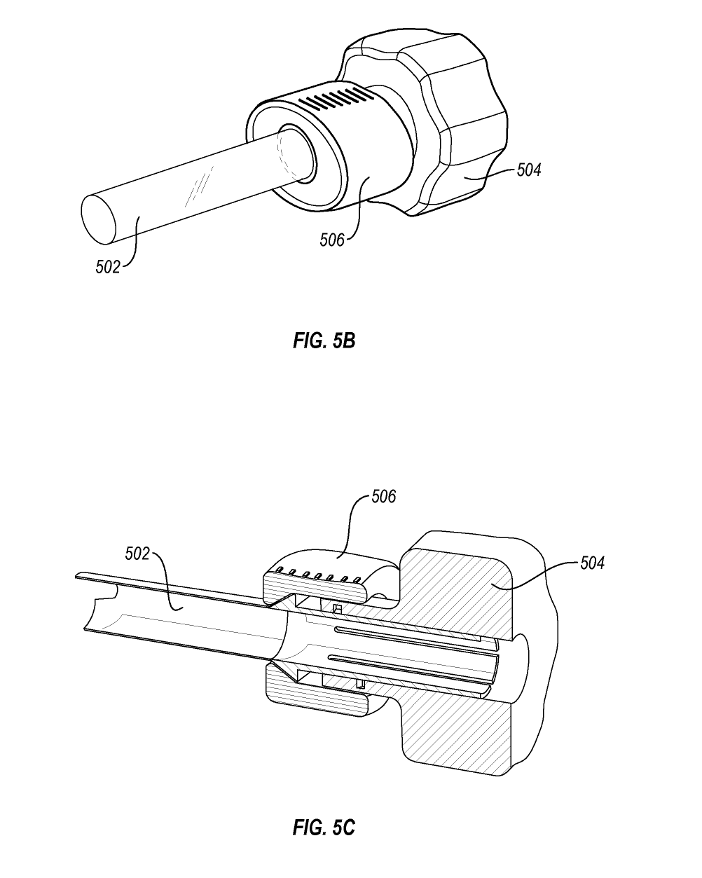

FIGS. 5A-5C illustrate an exemplary laparoscope cover 500 that includes a sheath in the form of an elongate cylindrical tube 502. A gripping hub portion 504 configured to facilitate gripping and placement of the laparoscope cover 500 over a laparoscope (not shown) is positioned at a proximal end of elongate tubular member 502. A strengthening hub portion 506 adjacent to the gripping hub portion 504 provides additional strength and rigidity to the proximal end of elongate cylindrical tube 502. The distal end 508 may include a nanoscopically and/or microscopically rough surface as disclosed herein so as to provide at least one of a highly hydrophobic, highly oleophobic, or highly hydrophilic surface. At least a portion of the outer sidewall of elongate cylindrical tube 502 may include the nanoscopically and/or microscopically rough surface. According to one embodiment, an outer ring of distal tip 508 may be devoid of the nanoscopically and/or microscopically rough surface to provide a location for preferential movement and adhesion of moisture or debris toward the outer ring and away from the observation window at the distal tip 508 as a way to maintain clear and unobstructed vision. Alternatively, an outer ring of distal tip 508 may include a highly hydrophilic surface coating to provide a location for preferential movement and adhesion of moisture or debris toward the outer ring and away from the observation window at the distal tip 508 as a way to maintain clear and unobstructed vision.

The elongate tubular member of the laparoscope cover 500 can be blow molded from an appropriate polymer (e.g., PETG) that can accept a nanoscopically and/or microscopically rough surface as described herein or that may hereafter be developed or is already known in the art. The hub member can be directly molded over, or separately molded and then attached to, the proximal end of the elongate tubular member.

The exemplary laparoscope cover 500 can be held in place over a laparoscope during use by friction lock near the proximal end of the laparoscope handle. The fiber optics of the laparoscope will be partially or entirely disposed into the sheath. The sheath can be closed at the distal end to provide a sterile barrier to at least the distal end if not the entire laparoscope. In the case where the laparoscope is prone to getting very hot, the sheath can protect against fire (e.g., a patient surgical drape made of paper) and reduce the spread of heat inside the tip. This keeps the heat inside the sheath, which can reduce the temperature gradient between the laparoscope and the patient's body in order to reduce condensation on the sheath. The sheath can also act as a heat sink to distribute and dissipate heat to decrease the tendency of heat to be focused at the tip where light is emitted.

The suface coating on the laparoscope cover 500 provides nanoscale roughness, which maximizes the contact angle of a water droplet on the treated polymer surface. The “contact angle” refers to the angle of the tangent of the water droplet to the surface. A perfect sphere on a hard surface (e.g., marble on a table) would have a contact angle approaching or equaling 180°.

Through hydrophobic chemistry alone, it is estimated that the maximum possible contact angle of a water droplet is about 120°. However, nanoscale roughness provided by the nanoparticles reduces the contact surface area between the water droplet and the polymer surface and traps air. This creates a liquid-air interface having much lower friction and bonding attraction compared to a liquid-solid interface, thereby increasing the ability of the natural surface tension of the water droplet to form a more spherical droplet and adhere less to the polymer surface. According to one embodiment, the highly hydrophobic composition or coating is formulated so as to cause water or protein-based droplets to have a surface angle relative to the polymer surface of at least about 135°, preferably at least about 140°, and more preferably at least about 150°. In the case of blood, the contact angle would be expected to be somewhat lower because blood has lower surface tension than water.

Similar logic holds for an oleophobic surface. Through oleophobic chemistry alone, it is estimated that the maximum possible contact angle of an oil droplet or a similar non-polar substance is about 120°. However, nanoscale roughness provided by the nanoparticles coupled with oleophobic surface treatment reduces the contact surface area between the oil droplet and the polymer surface and traps air. This creates a liquid-air interface having much lower friction and bonding attraction compared to a liquid-solid interface, thereby increasing the ability of the natural surface tension of the oil droplet to form a more spherical droplet and adhere less to the polymer surface. According to one embodiment, the highly oleophobic composition or coating is formulated so as to cause oil or non-polar droplets to have a surface angle relative to the polymer surface of at least about 135°, preferably at least about 140°, and more preferably at least about 150°. However, in the case of oil droplets or droplets of other non-polar liquids, the contact angle would be expected to be somewhat lower that with water because oils and other non-polar liquids have lower surface tension than water.

In the case of a highly hydrophilic surface, the nanoscale roughness provided by the nanoparticles coupled with hydrophilic surface treatment is expected to “wick” water into the surface such that water and other aqueous liquids will form a thin, substantially uniform layer of water that covers the surface. Such a thin, substantially uniform layer of water can, for example, prevent fogging by water condensation on the surface.

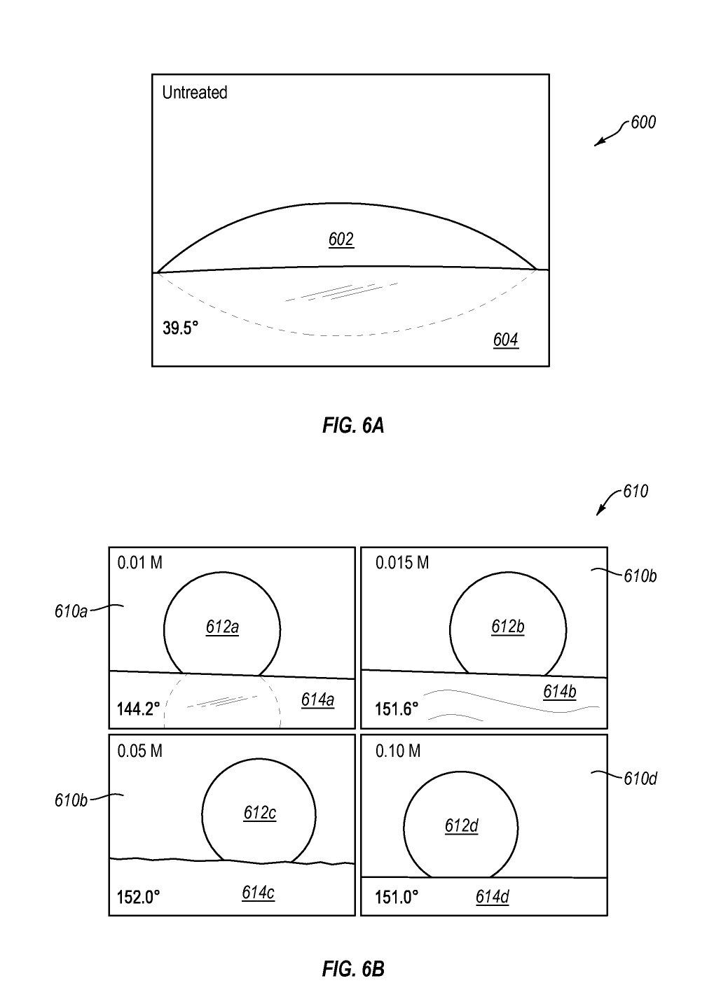

FIGS. 6A and 6B illustrate water droplets having different contact angles relative to a surface. FIG. 6A shows illustrative image 600 showing a water droplet 602 attached to an untreated surface 604 and having a low contact angle. FIG. 6B, by contrast, illustrates illustrative images 610 showing water droplets having high contact angles using superhydrophobic coatings within the disclosure. Illustrative image 610a shows a water droplet 612a on a first treated surface 614a with a contact angle of 144.2°. Illustrative image 610b shows a water droplet 612b on a second treated surface 614b with a contact angle of 151.6°. Illustrative image 610c shows a water droplet 612c on a third treated surface 614c with a contact angle of 152.0°. Illustrative image 610d shows a water droplet 612d on a fourth treated surface 614d with a contact angle of 151.0°.

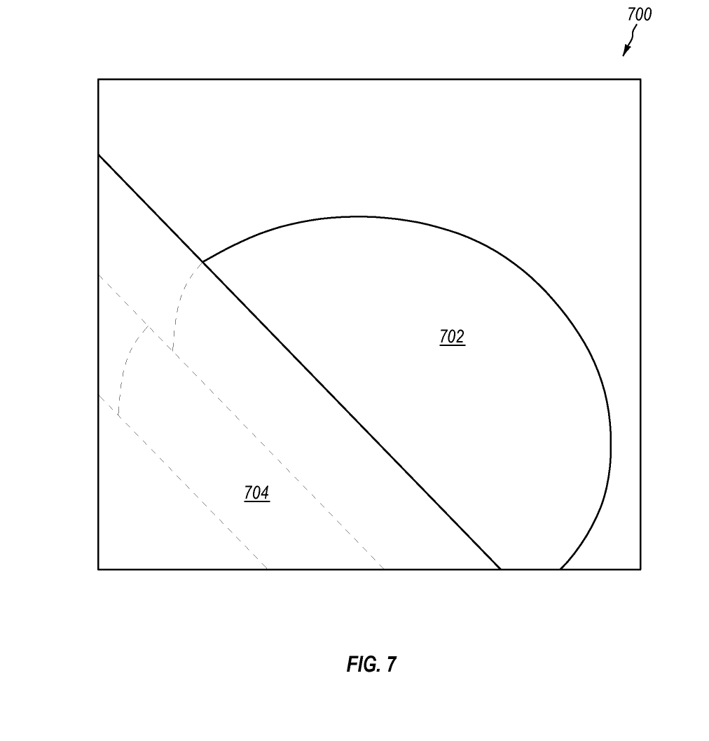

The highly hydrophobic composition is also formulated so as to reduce the sliding or hysteresis angle of a droplet of water on a polymer surface as much as possible. The “sliding angle” is the angle beyond level at which a droplet of water or blood runs off the highly hydrophobic surface. FIG. 7 is an illustrative image 700 showing how a water droplet 702 on surface 704 bulges under the force of gravity when the surface is held at an angle above level. The lower the sliding angle, the greater will be the tendency of the water or blood droplet to run off the surface, thereby preventing or reducing buildup of vision obscuring fog or condensation. According to one embodiment, the highly hydrophobic composition is also formulated so that the hysteresis angle of a droplet of water on a polymer surface is less than about 30°, preferably less than about 15°, more preferably less than about 10°, and most preferably less than about 5°.

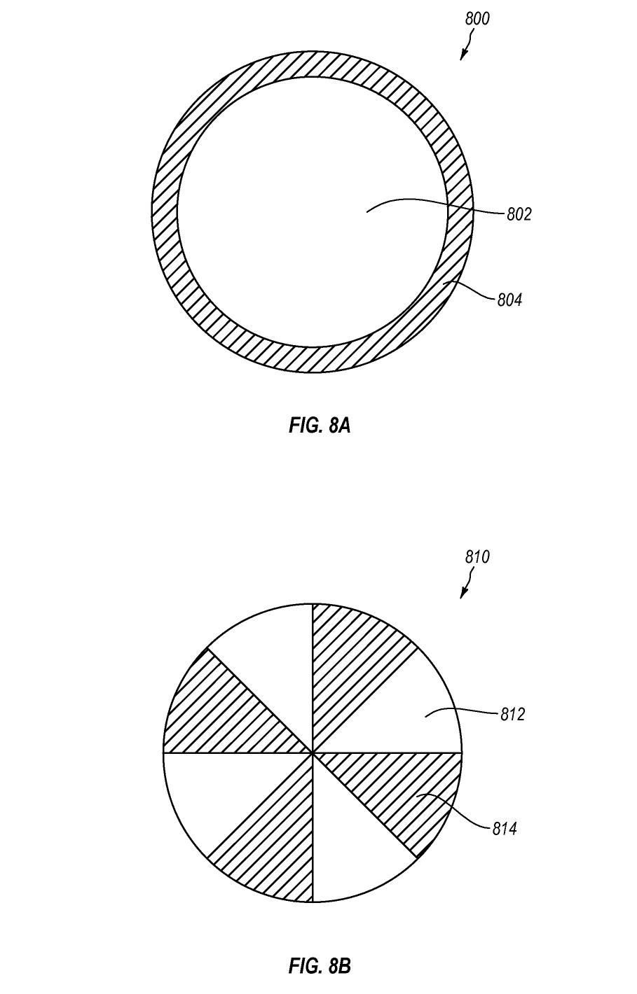

According to one embodiment, a composition may contain discrete regions of superhydrophobic and superhydrophilic materials. Superhydrophilic materials can be made in the same way as superhydrophobic materials by placing a superhydrophilic substrate on the outer surface of the composition. FIGS. 8A and 8B schematically illustrate exemplary embodiments of coatings or compositions designed to draw moisture away from and toward specific regions as a result of the interplay between the superhydrophobic and superhydrophilic regions.

FIG. 8A schematically illustrates a composition or coating 800 having a superhydrophobic region 802 comprised of a superhydrophobic material as described herein surrounded by a superhydrophilic region 804 around the perimeter of the composition or coating 800. In this embodiment, moisture will migrate from the superhydrophobic region 802 toward the superhydrophilic region 804 at the perimeter. This helps channel the moisture from a region where it is less desirable to have moisture (e.g., interior) to a region where it is more desirable and/or less deleterious to have moisture (e.g., perimeter).

FIG. 8B schematically illustrates a composition or coating 810 having a plurality of superhydrophobic regions 812 comprised of a superhydrophobic material as described herein separated by intervening superhydrophilic regions 814. In this embodiment, moisture will migrate from the superhydrophobic regions 812 toward the superhydrophilic regions 814. This arrangement facilitates movement of moisture away from the center of the composition.

Providing a laparoscope cover with a highly hydrophobic coating that provides a high contact angle and/or low hysteresis angle of a droplet of water or blood on the surface maximizes visibility and light transmittance. These features increase light transmittance by preventing or reducing formation of a uniform fog layer over the laparoscope cover. According to one embodiment, the polymer material used to make the sheath of the laparoscope cover and the highly hypdrophobic coating are sufficiently transparent that light transmittance is not reduced by more than about 20%, and preferably less than 20%. The size of the nanoparticles also affects light transmittance. Nanoparticles larger than about 200 nm can cause light scattering, which can blur the view. Nanoparticles smaller than about 200 nm typically do not scatter light, which increases light transmittance and sharpness of the image produced by the laparoscope. According to one embodiment, the laparoscope cover and highly hydrophobic composition are configured so that the laparoscope cover does not decrease light transmittance from a laparoscope by more than about 20%.

The laparoscope cover may alternatively comprise a sheath with a hydrophobic coating thereon in the form of a flexible film (i.e., “band-aid”) (not shown) that can be applied to the observation window to provide the non-stick coating.

For purposes of this disclosure, the term “laparoscope” can be considered an “endoscope” as well. Both can also be termed a borescope. Accordingly, the foregoing description relative to applying a highly hydrophobic coating to a laparoscope cover can also be applicable to an endoscope cover for use with an endoscope for providing clearer and less obstructed vision at a surgical, diagnostic or procedure site. The endoscope cover may comprise a sheath configured for placement over at least a portion of an endoscope, at least a portion of the sheath being transparent to light, and a non-stick coating on at least a portion of the sheath that reduces or prevents adhesion of substances that obstruct vision at a surgical site.

An exemplary method of performing a laparoscopic procedure includes: (1) providing a cover configured to be fitted onto a laproscope, wherein the cover includes a nanoscopically and/or microscopically rough surface that may be at least one of a highly hydrophobic, highly oleophobic, or highly hydrophilic; (2) positioning the cover including the nanoscopically and/or microscopically rough surface adjacent to at least a portion of a laparoscope, wherein the nanoscopically and/or microscopically rough surface reduces or prevents adhesion of substances that obstruct vision; (3) positioning the laparoscope at a surgical site; and (4) utilizing the laparoscope to illuminate and view the surgical site, (5) the nanoscopically and/or microscopically rough surface reducing or preventing adhesion of substances that obstruct vision at the surgical site.

It will also be appreciated that the present claimed invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope. Additionally, as used in this specification and the appended claims, the singular forms “a,” “an” and “the” include plural referents unless the context clearly dictates otherwise.

1. A cover for use with a surgical viewing instrument for providing clearer and less obstructed vision at a surgical, diagnostic or procedure site, comprising:
a cover configured for placement over at least a portion of a surgical viewing instrument, at least a portion of the cover being transparent to light; and
a nanoscopically and/or microscopically rough surface formed on at least a portion of the cover that provides a clearer and less obstructed view of a surgical, diagnostic or procedure site through the surgical viewing instrument.

2. The cover of claim 1, wherein the nanoscopically and/or microscopically rough surface reduces or prevents adhesion of substances that obstruct vision at a surgical site.

3. The cover of claim 1, wherein the nanoscopically and/or microscopically rough surface is at least one of a highly hydrophobic, highly oleophobic, or highly hydrophilic surface.

4. The cover of claim 1, wherein the nanoscopically and/or microscopically rough surface comprises a coating applied to at least a portion of the cover.

5. The cover of claim 1, wherein the surgical viewing instrument is selected from the group consisting of a laparoscope, an endoscope, a boroscope, a capsule endoscope, a pill camera, or a surgical microscope.

6. The cover of claim 1, wherein the cover includes an elongate tubular member that at least partially encloses the surgical viewing instrument during use.

7. The cover of claim 6, wherein the nanoscopically and/or microscopically rough surface is positioned on at least a distal tip of the sheath.

8. The cover of claim 6, wherein the nanoscopically and/or microscopically rough surface is positioned on at least a sidewall of the sheath.

9. The cover of claim 6, wherein the elongate tubular member further includes one of an elastic region configured to secure the elongate tubular member to the surgical viewing instrument or a rigid hub attached to a proximal end of the elongate tubular member configured to secure the elongate tubular member to the surgical viewing instrument.

10. The cover of claim 1, wherein the cover comprises a flexible or shrinkable film configured to be applied to at least a portion of a surgical viewing instrument and wherein the nanoscopically and/or microscopically rough surface is positioned on at least portion of the flexible film.

11. The cover of claim 1, wherein the nanoscopically and/or microscopically rough surface is configured to reduce or prevent adhesion of blood, tissue debris and condensation on a surface of the cover.

12. The cover of claim 1, wherein the at least a portion of the nanoscopically and/or microscopically rough surface formed on the cover is a superhydrophilic surface configured to do at least one of wick aqueous liquids away from a superhydrophobic portion of the cover or promote formation of a substantially uniform layer of water on at least a portion of the cover.

13. The cover of claim 1, wherein a portion of the cover does not include the nanoscopically and/or microscopically rough surface to provide a location for preferential adhesion of substances to the cover that obstruct vision.

14. The cover of claim 1, wherein the at least a portion of the nanoscopically and/or microscopically rough surface formed on the cover comprises a highly hydrophobic composition that repels water and other hydrophilic substances.

15. The cover of claim 14, wherein the highly hydrophobic composition comprises nanoparticles held to the cover by one or more types of adhesion molecules.

16. The cover of claim 15, wherein the adhesion molecules are at least one of silanes or siloxanes.

17. The cover of claim 15, wherein the highly hydrophobic composition further comprises a hydrophobic surface modifying agent.

18. The cover of claim 17, wherein the hydrophobic surface modifying agent comprises at least one of fluoroalkyl or silane molecules.

19. The cover of claim 17, wherein a portion of the highly hydrophobic composition further comprises a hydrophilic surface modifying agent for preferential adhesion of water or other hydrophilic substances to one or more regions of the highly hydrophobic composition.

20. The cover of claim 14, wherein the highly hydrophobic composition is formulated so as to cause water-based droplets to have a surface angle of at least about 135° relative to a surface of the cover that includes the highly hydrophobic composition.

21. The cover of claim 14, wherein the highly hydrophobic composition is formulated so as to cause water-based droplets to have a surface angle of at least about 140° relative to a surface of the cover that includes the highly hydrophobic composition.

22. The cover of claim 14, wherein the highly hydrophobic composition is formulated so as to cause water-based droplets to have a surface angle of at least about 150° relative to a surface of the cover that includes the highly hydrophobic composition.

23. The cover of claim 14, wherein the highly hydrophobic composition is formulated so as to cause water-based droplets to have a shedding angle of less than about 30° relative to a surface of the cover that includes the highly hydrophobic composition.

24. The cover of claim 14, wherein the highly hydrophobic composition is formulated so as to cause water-based droplets to have a shedding angle of less than about 15° relative to a surface of the cover that includes the highly hydrophobic composition.

25. The cover of claim 1, wherein the at least a portion of the nanoscopically and/or microscopically rough surface formed on the cover is formulated so that the cover does not decrease light transmittance through the cover by more than about 20%.

26. An endoscope cover for use with an endoscope for providing clearer and less obstructed vision at a surgical, diagnostic, or procedure site, comprising:
a sheath configured for placement over at least a portion of an endoscope, at least a portion of the sheath being transparent to light; and
a nanoscopically and/or microscopically rough surface formed on at least a portion of the sheath that provides a clearer and less obstructed view of a surgical, diagnostic or procedure site through the surgical viewing instrument.

27. A method of performing a laparoscopic procedure comprising:
positioning a nanoscopically and/or microscopically rough surface on at least a viewing and illumination portion of a surgical viewing instrument;
positioning the surgical viewing instrument at a surgical site; and
utilizing the surgical viewing instrument to illuminate and view the surgical site,
the nanoscopically and/or microscopically rough surface reducing or preventing adhesion of substances that obstruct vision at the surgical site.

28. A method as in claim 27, wherein positioning the nanoscopically and/or microscopically rough surface on the surgical viewing instrument comprises placing a sheath carrying the nanoscopically and/or microscopically rough surface over at least a portion of the surgical viewing instrument.

29. A method as in claim 27, wherein positioning the nanoscopically and/or microscopically rough surface on the surgical viewing instrument comprises placing a transparent film carrying the nanoscopically and/or microscopically rough surface over at least a portion of the surgical viewing instrument.

30. A method as in claim 27, wherein positioning the nanoscopically and/or microscopically rough surface on the surgical viewing instrument comprises placing an elongate tubular member carrying the nanoscopically and/or microscopically rough surface over at least a portion of the surgical viewing instrument.

31. A method as in claim 30, wherein the elongate tubular member includes a hub at a proximal end that facilitates gripping and positioning of the elongate tubular member.

32. A method of manufacturing a cover for use with a surgical viewing instrument for providing clearer and less obstructed vision at a surgical site, comprising:
providing a polymeric member configured for placement over at least a portion of a surgical viewing instrument, at least a portion of the cover being transparent to light; and
forming a nanoscopically and/or microscopically rough surface on at least a portion of the polymeric member, wherein the nanoscopically and/or microscopically rough surface reduces or prevents adhesion of substances that obstruct vision at a surgical site.

33. A method as in claim 32, wherein the polymeric member comprises an elongate tubular member formed from a polymer and configured to at least partially enclose a laparoscope during use.

34. A method as in claim 32, wherein forming the nanoscopically and/or microscopically rough surface comprises:
reacting an organic binder with functional groups on a polymer surface of the polymeric member to bond organic binder molecules to the polymer surface;
reacting nanoparticles with the organic binder molecules; and
reacting a cross-linking agent with the nanoparticles to form cross-linked nanoparticles.

35. A method as in claim 34, wherein forming the nanoscopically and/or microscopically rough surface further comprises:
activating the polymer surface to form or expose the functional groups on the polymer surface prior to reacting the organic binder with the functional groups;
activating the organic binder molecules prior to reacting the nanoparticles with the organic binder molecules; and
applying a surface modifying agent to the cross-linked nanoparticles.

36. A method as in claim 32, wherein forming the nanoscopically and/or microscopically rough surface comprises:
forming a nanoscopically and/or microscopically roughened surface on at least one surface of a mold;
molding the polymeric member in the mold, wherein, in the molding, the nanoscopically and/or microscopically rough surface is imprinted on the polymeric member.

37. A method of forming a nanoscopically and/or microscopically rough surface on a polymer surface, comprising:
activating a polymer surface to yield a functionalized polymer surface having functional groups;
treating the functionalized polymer surface with an organic binder to yield a modified polymer surface having organic binder molecules bonded thereto;
coating the modified polymer surface with one or more of nanoparticles or microparticles to form a particle treated polymer surface;
reacting a cross-linking agent with the particle treated polymer surface to form cross-linked particle treated polymer surface; and
applying a functionalizing agent to the cross-linked particle treated polymer surface to yield the nanoscopically and/or microscopically rough coating on the polymer surface.

38. The method of claim 37, wherein the nanoscopically and/or microscopically rough surface is at least one of a highly hydrophobic, highly oleophobic, or highly hydrophilic surface.

39. The method of claim 37, wherein the polymer surface forms at least a portion of an article selected from the group consisting of ski goggles, swimming goggles, glasses, windows, vehicle windshields, motorcycle fairings, camera lenses, waterproof enclosures for cameras or other viewing equipment, endoscopes, smartphone surfaces, and tablet computer surfaces.

40. The method of claim 37, wherein the polymer surface comprises at least one polymer selected from the group consisting of polycarbonates, polyethylene terephthalate glycol modified (PETG), and polystyrene.

41. The method of claim 37, wherein the polymer surface is activated using plasma activation.

42. The method of claim 37, wherein the polymer surface is activated using at least one of a solvent, oxidizer, acid, or base.

43. The method of claim 37, wherein the functional groups on the functionalized polymer surface are selected from the group consisting of hydroxyl groups, carboxyl groups, amino groups, halide groups, sulfonyl groups, and combinations thereof.

44. The method of claim 37, wherein the organic binder comprises 3-(aminopropyl)triethoxy silane (APTES) substituted with at least one of an ethyl, propyl, butyl or higher alkyl.

45. The method of claim 44, wherein steric hindrance prevents formation of Si—O—C bonds with the polymer surface and favors formation of amine or amide bonds.

46. The method of claim 44, the method comprising reacting the functionalized polymer surface with a reaction mixture that includes APTES, water and an acid catalyst.

47. The method of claim 46, further comprising drying the modified polymer surface to remove water prior to coating the modified polymer surface with nanoparticles.

48. The method of claim 37, wherein the nanoparticles react with the organic binder molecules by displacing one or more leaving groups.

49. The method of claim 48, wherein the organic binder molecules include silane molecules and wherein the nanoparticles form Me-O—Si bonds with the silane molecules.

50. The method of claim 48, further comprising drying the nanoparticle treated polymer surface prior to reacting the cross-linking agent with the nanoparticle treated polymer surface.

51. The method of claim 37, wherein the cross-linking agent comprises a dipodal silane, such as bis-triethoxy-silyl ethane (BTESE).

52. The method of claim 37, further comprising drying the cross-linked nanoparticle treated polymer surface prior to applying the functionalizing agent to the cross-linked nanoparticle treated polymer surface.

53. The method of claim 37, wherein the functionalizing agent comprises methyltriethoxysilane (MTES).

54. The method of claim 37, wherein the functionalizing agent comprises fluoroalkyl groups to provide a coating that is both hydrophobic and oleophobic.

55. The method of claim 37, wherein a portion of the cross-linked nanoparticle treated polymer surface is treated with a hydrophilic functionalizing agent to provide hydrophilic properties in one or more regions.

56. The method of claim 55, wherein the hydrophilic functionalizing agent is a polyethylene glycol (PEG).

57. A method for forming a superhydrophobic, superoleophobic, and/or superhydrophilic surface or combination thereof comprises:
(1) activating a substrate to improve chemical bonding;
(2) depositing an adhesion promoter;
(3) depositing at least one of nanoparticles or microparticles to said surface to create a nanoscopically and/or microscopically rough surface;
(4) crosslinking the nanoparticles or microparticles with a crosslinking agent; and
(5) covalently bonding at least one of a hydrophilic, hydrophobic, or oleophobic material to the crosslinking agent to yield a nanoscopically and/or microscopically rough having one or more of a hydrophilic, hydrophobic, or oleophobic surface characteristic.

58. A nanoscopically and/or microscopically rough coating on a polymer surface, comprising:
a functionalized polymer surface;
an organic binder bonded to the functionalized polymer surface;
nanoparticles bonded to the polymer surface by means of the organic binder;
a cross-linking agent bonded to the nanoparticles to form a cross-linked nanoparticle treated polymer surface; and
a functionalizing agent bonded to the cross-linked nanoparticle treated polymer surface that imparts at least one of a superhydrophobic, superhydrophilic, or superoleophobic surface coating on the polymer surface.

59. The nanoscopically and/or microscopically rough coating on a polymer surface of claim 58, wherein the cross-linking agent comprises a dipodal silane, such as bis-triethoxy-silyl ethane (BTESE).

60. The nanoscopically and/or microscopically rough coating on a polymer surface of claim 58, wherein the functionalizing agent comprises methyltriethoxysilane (MTES).

61. The nanoscopically and/or microscopically rough coating on a polymer surface of claim 58, wherein the functionalizing agent comprises fluoroalkyl groups to provide a coating that is both hydrophobic and oleophobic.

62. The nanoscopically and/or microscopically rough coating on a polymer surface of claim 58, wherein a portion of the superhydrophobic coating further comprises a hydrophilic surface modifying agent for preferential adhesion of water or other hydrophilic substances to one or more regions of the superhydrophobic coating.

63. The nanoscopically and/or microscopically rough coating on a polymer surface of claim 58, wherein the nanoscopically and/or microscopically rough coating is formulated to not decrease light transmittance by more than about 20%.
